(19) United States
(12) Patent Application Publication
(10) Pub. No.: US 2023/0326691 A1
(43) Pub. Date: Oct. 12, 2023

(54) MEDICAL TECHNOLOGY DEVICE WITH WIRELESSLY CONNECTED FOOT SWITCH

(71) Applicant: OLYMPUS WINTER & IBE GMBH, Hamburg (DE)

(72) Inventor: Michael WOLTER, Hamburg (DE)

(73) Assignee: OLYMPUS WINTER & IBE GMBH, Hamburg (DE)

(21) Appl. No.: 18/132,203

(22) Filed: Apr. 7, 2023

Related U.S. Application Data
(60) Provisional application No. 63/329,735, filed on Apr. 11, 2022.

(51) Int. Cl. H01H 13/16; A61B 18/00

(52) U.S. Cl. CPC H01H 13/16; A61B 2017/00973; A61B 18/00

(57) ABSTRACT

The invention relates to a system (1) comprising a medical technology device (10) controllable via a foot switch (20), and a foot switch (20), wherein the foot switch (20) comprises a transmitter and the medical technology device (10) comprises a receiver for the wireless transmission of unambiguously assignable signals for the control from the foot switch (20) to the medical technology device (10), and wherein the receiver of the medical technology device (10) is designed to measure the signal strength of signals from the foot switch (20), and the medical technology device (10) is designed to emit a warning if the measured signal strength or the signal-to-noise ratio lies below a predefined threshold value.

The invention relates to a system comprising a medical technology device controllable via a foot switch, and a foot switch.

Foot switches are commonly used in medical technology to control medical technology devices. Foot switches can be used, for example, to control the activation state of a hand-guided instrument without the guiding hand itself, or even only one finger thereof, having to be moved. Medical staff can therefore concentrate solely on the guidance of the instrument by hand and can control the activation state of the instrument by foot separately therefrom. The activation state does not have to be restricted to the activation and deactivation of a specific function of the hand-guided instrument, but can also comprise stepless adjustability. Examples of medical technology devices with corresponding foot control are dental instruments such as drills, HF generators for high-frequency surgery, ultrasonic generators for ultrasonic scalpels and/or laser systems.

Foot switches can be connected by cable to the medical technology device which is to be controlled. However, in order to avoid restricting or hindering the freedom of movement of medical staff, for example in an operating theater, foot switches are also known which transmit control signals wirelessly to the medical technology device which is to be controlled. A suitable receiver for the control signals must then either be integrated directly into the medical technology device, or a suitable external receiver is to be provided at the signal output, to which, for example, a cable-connected foot switch can also be connected.

In order to ensure trouble-free use of a medical technology device controllable via a foot switch, it is necessary, in the case of wireless transmission of control signals, for the signal strength of the control signal received on the medical technology device is sufficiently high so that the control signals can be unambiguously identified and can then be implemented.

Evidence has shown that, in cases where medical technology devices are used, for example in surgeries or operating theatres, various interference sources can affect the wireless transmission of control signals between a foot switch and the assigned medical technology device, said interference sources restricting or even totally preventing the correct operation of a medical technology device via the foot switch.

Possible interference sources are other devices transmitting radio signals, which either transmit at the same frequency and therefore overlay the control signals, or which at least cause increased noise from which the control signals are no longer sufficiently distinguishable. Mobile instrument tables which are often made of metal, or rollable technical devices can also interfere with the radio path between the foot switch and the assigned medical technology device.

In particular, interference occurring during the essential use of the medical technology device, for example due to the movement of instrument tables or the activation of other technical devices, can be critical. Extensive troubleshooting is also not usually possible in such situations—often during the treatment of a patient.

The object of the present invention is to provide a system comprising a medical technology device controllable via a foot switch, and a foot switch in which the problems known from the prior art are at least alleviated.

This object is achieved by a system as claimed in the main claim. Advantageous developments form the subject-matter of the dependent claims.

The invention thus relates to a system comprising a medical technology device controllable via a foot switch, and a foot switch, wherein the foot switch comprises a transmitter and the medical technology device comprises a receiver for the wireless transmission of unambiguously assignable signals for the control from the foot switch to the medical technology device, wherein the receiver of the medical technology device is designed to measure the signal strength of signals from the foot switch and the medical technology device is designed to output a warning if the measured signal strength or the signal-to-noise ratio lies below a predefined threshold value.

The invention has recognized that, by monitoring the signal strength of the signals transmitted by the foot switch or its transmitter, it can at least be ensured on the medical technology device that interference suddenly occurring during the use of the medical technology device and affecting the radio link between the foot switch and the medical technology device can be immediately identified as such. Moreover, a corresponding emitted warning often enables an immediate determination of the interference source, such as, for example, the movement of an instrument table or the activation of a specific device, to which a suitable response can then be given, for example by moving the instrument table, the device in question or the foot switch in order to reduce the interference to the extent that the signal strength is sufficient for interference-free operation.

Even if totally interference-free communication between the foot switch and the medical technology device cannot therefore be ensured by means of the invention independently from external circumstances, a warning is nevertheless emitted immediately in the system according to the invention as soon as interference occurs which affects the communication concerned. This enables immediate intervention, for example to eliminate the interference source, thereby preventing a failure which would otherwise occur suddenly for the user of the medical technology device and would not be immediately explicable. A failure of this type is highly undesirable in the case of medical technology devices and their normal use in operating theatres or treatment rooms, and can even have significant consequences for the health of a patient.

Alternatively or additionally to the determination of the actual signal strength and the comparison with a predefined threshold value, the signal-to-noise ratio for the signals from the foot switch can also be monitored. The signal-to-noise ratio indicates how clearly the useful signal, i.e. here the signal from the foot switch, is distinguishable from background noise, which has a direct impact on the ability to extract information reliably from the signal. In the case of digital signals, a relationship often exists between the signal-to-noise ratio and the error rate.

It is preferred if the transmitter of the foot switch and the receiver of the medical technology device are in each case part of a transceiver in each case comprising a transmitter and a receiver for two-way communication between the foot switch and the medical technology device. This enables two-way communication between the foot switch and the medical technology device which can be used, for example, to couple the footswitch with the medical technology device, i.e. to establish a logical link between the foot switch and the medical technology device in such a way that only the signals from the foot switch are processed only by the medical technology device concerned.

If transceivers are provided in both the foot switch and the medical technology device, it is preferred if the transceiver of the foot switch is used to measure the signal strength of signals from the medical technology device and the foot switch is used to emit a warning if the measured signal strength or the signal-to-noise ratio lies below a predefined threshold value. In this way, the signal strength and/or the signal-to-noise ratio and therefore the interference-free communication between the foot switch and the medical technology device can be checked redundantly, so that the certainty of being alerted immediately to interference affecting this communication is increased. A malfunction of one of the two transceivers can therefore also be identified as possible interference affecting the communication. In such a case, for example, only one of the two devices would in fact emit a warning.

It can be advantageous if the transceiver of the foot switch and/or of the medical technology device is designed to transmit a signal in response to a ping signal received from the transceiver of the medical technology device or of the foot switch, on the basis of which the receiver of the medical technology device or of the foot switch can determine the signal strength. A “ping signal” is a signal transmitted for the purpose of receiving a return response from the communication partner which is normally without specific content and merely enables measurement of the signal strength according to the invention and/or the signal-to-noise ratio. It is obviously also possible to determine further information, such as, for example, signal round-trip delay times, from the return response to a ping signal.

Alternatively or additionally, it is further preferred if the foot switch and/or the medical technology device is designed to regularly transmit signals independently from a required control of the medical technology device, on the basis of which the receiver of the medical technology device or of the foot switch can determine the signal strength.

With both of the above-mentioned variants, it can be ensured that signals are exchanged between the foot switch and the medical technology device even if, for example due to a non-actuation of the foot switch, no actual control signals have to be transmitted. In the described variant with the ping signal, the electronics of the one device which merely has to respond to the ping signal can often be designed as very simple, which is appropriate for the foot switch in particular. The described regular transmission of signals has proven to be particularly robust.

The foot switch and/or the medical technology device can have visual and audible warning elements for the emission of a warning. An audible warning element, in particular, is preferred for the foot switch, whereas the medical technology device preferably has both visual and audible warning elements. A visual warning element can, for example, be a warning light or a warning LED; the audible warning element can be a tone generator.

It is alternatively or additionally preferred if the foot switch and/or the medical technology device has a visual indicator to display the signal strength. The visual indicator can be designed, for example, as a bar display of the type known from cell phones. A corresponding indicator can help to highlight problems in the communication between the foot switch and the medical technology device before actual interference occurs which results in a warning.

The foot switch can comprise an energy store, preferably a battery, for totally wireless operation.

The foot switch and the medical technology device are preferably designed for communication according to a Bluetooth standard.

The medical technology device which is to be controlled can, for example, be an HF generator, in particular for endoscopy, an ultrasonic generator and/or a laser system.

The invention will now be further described by way of example on the basis of an advantageous embodiment with reference to the attached drawing, In the drawing:

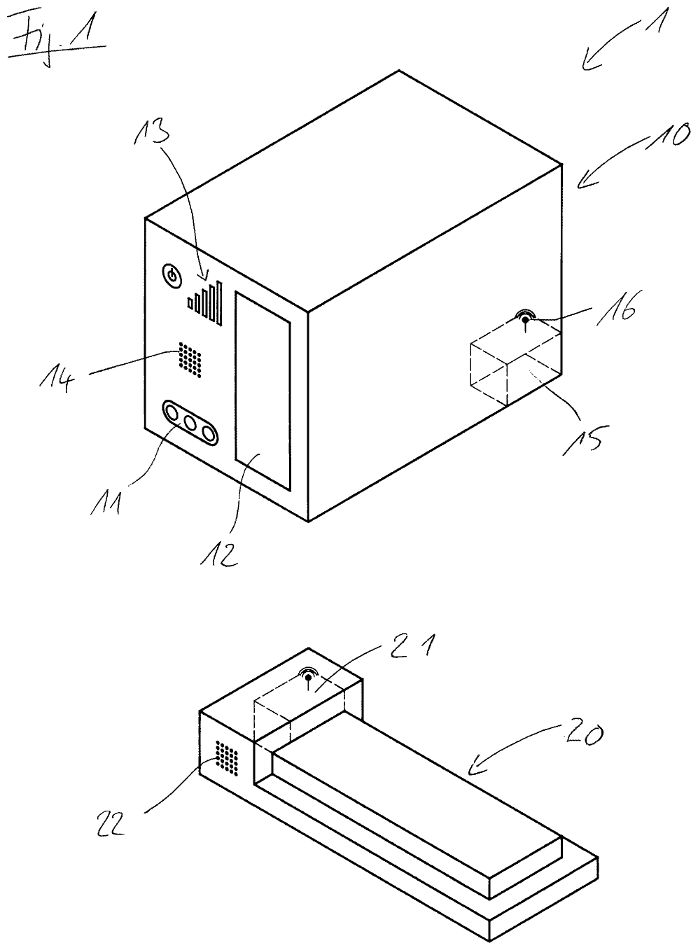

FIG. 1: shows a first exemplary embodiment of a system according to the invention.

FIG. 1 shows a first exemplary embodiment of a system 1 according to the invention comprising a medical technology device 10 and a foot switch 20 which are wirelessly interconnected.

The medical technology device 10 is an HF generator to the terminals 11 of which an electroscalpel (not shown) can be connected, for example for minimally invasive surgery also. The settings for the HF generator can be read from, but can also defined on, the touch-sensitive display 12.

A bar display 13 and a tone generator as the audible warning element 14 are provided along with the display 12. Internally within the medical technology device 10, a transceiver 15 with a similarly internal antenna 16 is provided. The transceiver comprises a transmitter and a receiver and is designed to transmit and receive signals according to a Bluetooth standard.

The foot switch 20 is battery-operated and similarly has a transceiver 21 which is designed to transmit and receive signals according to the same Bluetooth standard as the transceiver 15 of the medical technology device 10.

Furthermore, the foot switch 20 also has a tone generator as an audible warning element 22.

Both the transceiver 15 of the medical technology device 10 and the transceiver 21 of the foot switch 20 are designed to communicate with one another. Signals are transmitted, in particular, via the actuations of the foot switch 20 between these two transceivers 15, 21 so that the medical technology device 10 is controlled according to the setting of the foot switch 20.

Both transceivers 15, 21 are further designed to determine the signal strength and the signal-to-noise ratio of the signals exchanged between them. The signal strength determined by the transceiver 15 of the medical technology device is displayed directly via the bar display 13.

In addition, both the transceiver 15 of the medical technology device 10 and the transceiver 21 of the foot switch 20 compare the determined signal strength and the determined signal-to-noise ratio in each case with predefined threshold values. If a threshold value is understepped, this indicates an interrupted or poor connection between the two transceivers 15, 21, whereupon an audible warning is emitted via the respective tone generator 14, 22. In addition, the bar display 13 of the medical technology device 10 can be illuminated and/or can flash in a color differing from the usual color.

In order to ensure that the transceiver 21 of the foot switch can regularly perform the relevant determination and checking of the signal strength or the signal-to-noise ratio, the transceiver 15 of the medical technology device 10 transmits signals at regular intervals to the foot switch 20, said signals being received and processed accordingly by the transceiver 21 of the foot switch 20.

These signals regularly transmitted by the transceiver 15 of the medical technology device 10 are ping signals which, along with the checking of the signal strength or the signal-to-noise ratio by the foot switch 20, simultaneously represent a prompt to the transceiver 21 of the foot switch 20 to similarly transmit a signal, irrespective of whether an actuation of the foot switch, which would alternatively trigger a signal, takes place or not. In this way, the transceiver 15 of the medical technology device 10 can also regularly check the signal strength and the signal-to-noise ratio, and, as described, can emit a warning as soon as one of the two values drops below a respectively predefined threshold value.

The above-mentioned signals can similarly be, in particular, ping signals provided in the Bluetooth standard that is used.

1. A system (1) comprising a medical technology device (10) controllable via a foot switch (20), and a foot switch (20), wherein the foot switch (20) comprises a transmitter and the medical technology device (10) comprises a receiver for the wireless transmission of unambiguously assignable signals for the control from the foot switch (20) to the medical technology device (10),
characterized in that
the receiver of the medical technology device (10) is designed to measure the signal strength of signals from the foot switch (20), and the medical technology device (10) is designed to emit a warning if the measured signal strength or the signal-to-noise ratio lies below a predefined threshold value.

2. The system as claimed in claim 1,
characterized in that
the transmitter of the foot switch (20) and the receiver of the medical technology device (10) are in each case part of a transceiver (15, 20) in each case comprising a transmitter and a receiver for two-way communication between the foot switch (20) and the medical technology device (10).

3. The system as claimed in claim 2,
characterized in that
the transceiver (21) of the foot switch (20) is designed to measure the signal strength of signals from the medical technology device (10), and the foot switch (20) is designed to emit a warning if the measured signal strength or the signal-to-noise ratio lies below a predefined threshold value.

4. The system as claimed in claim 2,
characterized in that
the transceiver (21, 15) of the foot switch (20) and/or of the medical technology device (10) is designed to transmit a signal in response to a ping signal received from the transceiver (15, 21) of the medical technology device (10) or of the foot switch (20), on the basis of which the receiver of the medical technology device (10) or of the foot switch (20) can determine the signal strength.

5. The system as claimed in claim 1,
characterized in that
the foot switch (20) and/or the medical technology device (10) is designed to regularly transmit signals independently from a required control of the medical technology device, on the basis of which the receiver of the medical technology device (10) or of the foot switch (20) can determine the signal strength and/or the signal-to noise ratio.

6. The system as claimed in claim 1,
characterized in that
the foot switch (20) and/or the medical technology device (10) has visual and/or audible warning elements (14, 22) to emit a warning.

7. The system as claimed in claim 1,
characterized in that
the foot switch (20) and/or the medical technology device (10) has a visual indicator (13) to display the signal strength.

8. The system as claimed in claim 1,
characterized in that
the foot switch (20) comprises an energy store, preferably a battery, for wireless operation.

9. The system as claimed in claim 1,
characterized in that
the foot switch (20) and the medical technology device (10) are designed for communication according to a Bluetooth standard.

10. The system as claimed in claim 1,
characterized in that
the medical technology device (10) which is to be controlled is an HF generator, in particular for endoscopy, an ultrasonic generator and/or a laser system.
